(19) United States
(12) Patent Application Publication
(10) Pub. No.: US 2007/0014684 A1
(43) Pub. Date: Jan. 18, 2007

(54) Air purification system

(76) Inventors: Wayne A. Case, Portland, OR (US); Levi New, Kalamazoo, MI (US)

Correspondence Address: John R. Thompson;STOEL RIVES LLP, One Utah Center, 201 South Main Street, Suite 1100, Salt Lake City, UT, 84111, US

(21) Appl. No.: 11/168,270

(22) Filed: Jun. 28, 2005

Related U.S. Application Data
(63) Continuation-in-part of application No. 10/706,240, filed on Nov. 12, 2003, now Pat. No. 7,059,550.
(60) Provisional application No. 60/584,540, filed on Jul. 1, 2004.

(51) Int. Cl. A61L 9/16

(52) U.S. Cl. 422/4; 422/120; 422/127

(57) ABSTRACT

An airflow generator, coupled to a venturi, generates a high speed airflow to deliver air through a venturi. The air includes biological cells which are sterilized as the air passes through the venturi. Operation of the airflow generator may be controlled by a computer that responds to an indicated biological hazard.

### RELATED APPLICATIONS

This utility application claims priority to U.S. provisional patent application No. 60/584,540 filed on Jul. 1, 2004 and entitled “Air Purification Apparatus” and to U.S. utility patent application Ser. No. 10/706,240 filed on Nov. 12, 2003 and entitled “System and Method for Pulverizing and Extracting Moisture,” both of which are incorporated herein by reference.

### TECHNICAL FIELD

The aspects disclosed herein relate to air purifiers and, more specifically, to systems that purify air by destroying cells.

### BRIEF DESCRIPTION OF THE DRAWINGS

A more particular description of the invention briefly described above will be rendered by reference to the appended drawings. Understanding that these drawings only provide information concerning typical embodiments of the invention and are not therefore to be considered limiting of its scope, the invention will be described and explained with additional specificity and detail through the use of the accompanying drawings, in which:

FIG. 1 is a diagram of an air purification system.

FIG. 2 is a diagram of an alternative air purification system.

FIG. 3 is a diagram of an alternative air purification system.

FIG. 4 is a diagram of an alternative air purification system.

### DETAILED DESCRIPTION OF PREFERRED EMBODIMENTS

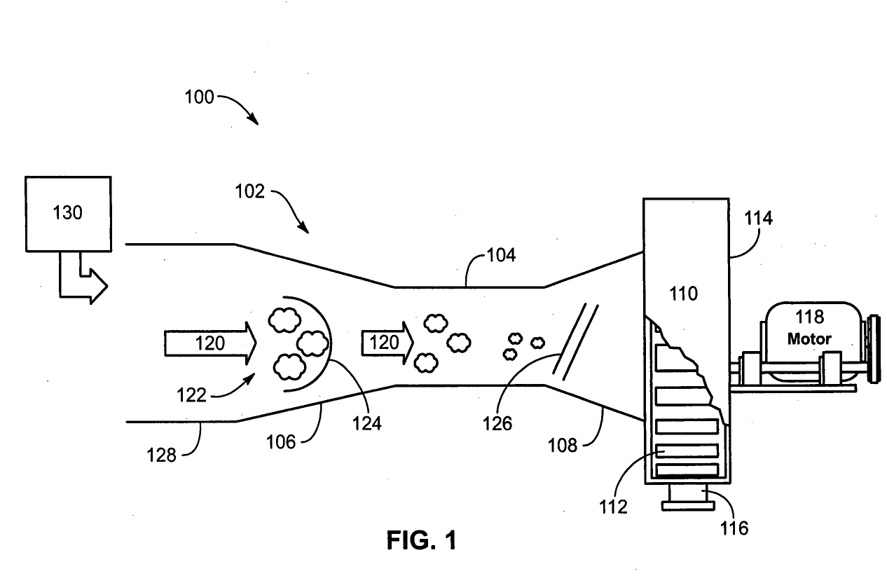

The presently preferred embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the apparatus, system, and method of the present invention, as represented in FIGS. 1 through 4, is not intended to limit the scope of the invention, as claimed, but is merely representative of presently preferred embodiments of the invention.

Referring to FIG. 1, a cross-sectional view of an air purification system 100 is shown. The air purification system 100 comprises a venturi 102 that includes a cylinder or throat 104. The venturi 102 further includes a converging portion 106 coupled to one end of the throat 104, and a diverging portion 108 is coupled to the opposing end of the throat 104. The venturi 102 functions to increase gas velocity and reduce gas pressure for a gas passing through.

An airflow generator 110 is in communication with the venturi 102. The airflow generator 110 may be embodied as a fan, impeller, turbine, a hybrid of a turbine and fan, a pneumatic suction system, or other suitable device for generating a high speed airflow. The airflow generator 110 may be directly coupled to the diverging portion 108. Alternatively, a cylindrical member may be coupled between the diverging portion 108 and the airflow generator 110. The airflow generator 110 may include a plurality of radially extending blades 112 that rotate to generate a high speed airflow. The blades 112 may be disposed within a housing 114 that includes a housing outlet 116 that provides an exit to incoming air. The housing 114 may couple to the venturi 102 and has a housing input aperture (not shown) that allows communication between the venturi 102 and the housing interior. The blades 112 define radially extending flow passages through which air passes to a housing outlet 116 to allow air to exit.

A motor 118 is coupled to the airflow generator 110 using known techniques to provide rotational movement. The motor 118 may be chosen from any one of a variety of suitable motors. The horse power of the motor 118 may vary significantly and depends on airflow rate and airflow generator dimensions.

In operation, the airflow generator 110 produces an airflow 120 that may range from 350 mph to supersonic. The airflow proceeds through the venturi 102 and into the airflow generator 110. Biological cells 122 within the airflow 120 encounter a first shock wave 124 as the cells 122 are introduced into the converging portion 106 of the venturi 102. The first shock wave 124 is created as the faster moving air encounters the slower moving air. As the cells 122 proceed into the venturi 102, they are subjected to extreme compression or, as referred to herein, hyper-compression due to increased pressure. The cells 122 further experience heat generated by friction. The compression and heat contributes to cell structure reduction and destruction.

The compressed cells 122 further encounter a second shock wave 126 as they enter the diverging portion 108. The second shock wave 126 obliterates remaining cell structure and ensures air sterilization. Thus, cells 122 encounter multiple forces from compression, friction, and shockwaves that create cell structure disruption.

The air purification system may further include an inlet tube 128 coupled to the venturi 102 to deliver incoming air. The air purification system 100 may further include a heat source 130 to generate and introduce heat into the converging portion 106 of the venturi 102. The heat further accelerates cell destruction and sterilization of the airflow 120. The heat generation may be varied based on perceived need and biological hazard. The heat source 130 may be directed into the inlet tube 128 or may feed directly into the converging portion 106.

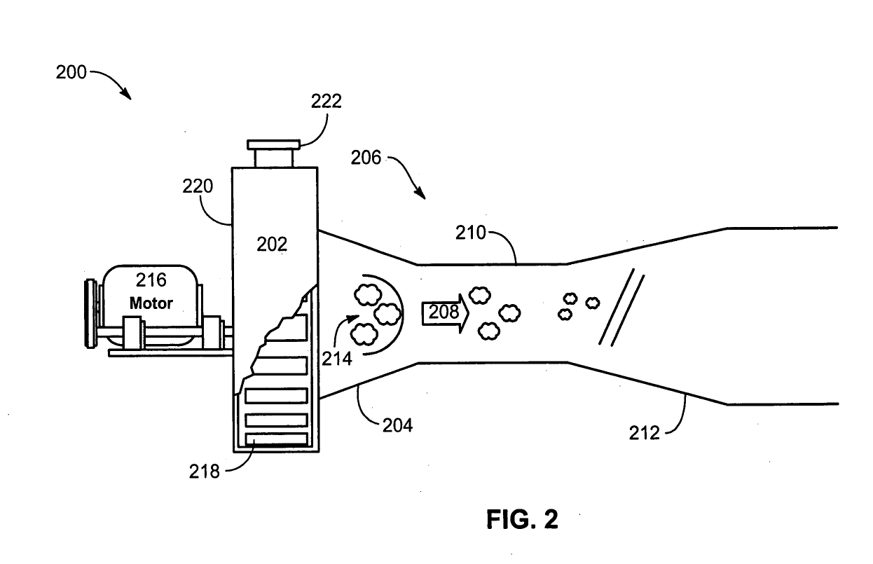

Referring to FIG. 2, an alternative embodiment of an air purification system 200 is shown. In the air purification system 200, an airflow generator 202 is coupled to a converging portion 204 of a venturi 206 to propel an airflow 208 through the venturi 206. The venturi 206 includes a throat 210 and a diverging portion 212 to operate in a similar manner as previously described. As with the previous embodiment, cells 214 are subjected to compression, heat, and shockwaves to disintegrate their structure and purify the air. The airflow generator 202 may be coupled to a motor 216 to provide radial movement of blades 218. The airflow generator 202 includes a housing 220 that coupled to an air inlet 222.

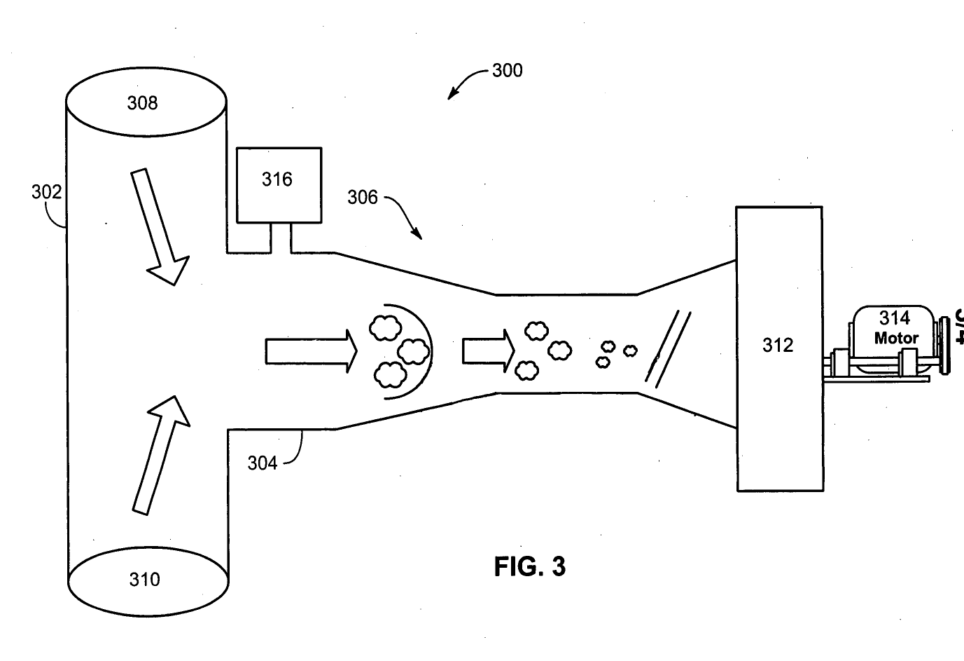

Referring to FIG. 3, an alternative embodiment of an air purification system 300 is shown which includes an inlet device 302 that couples to an inlet tube 304. The inlet device 302 may have a longitudinal axis that runs perpendicular to the longitudinal axis of a venturi 306 and inlet tube 304. The inlet device 302 may include opposing first and second apertures 308, 310 through which air passes. The inlet device 302 may be sized to accommodate and couple to a ventilation pipe. Valves may be disposed adjacent the apertures 308, 310 to control and direct airflow through the inlet device 302 as desired. As in previous embodiments, the system 300 includes an airflow generator 312, a motor 314 coupled to the airflow generator 312, and, in some implementations, a heat generator 316. The heat generator 316 may be in communication with the inlet tube 304 or the venturi 306.

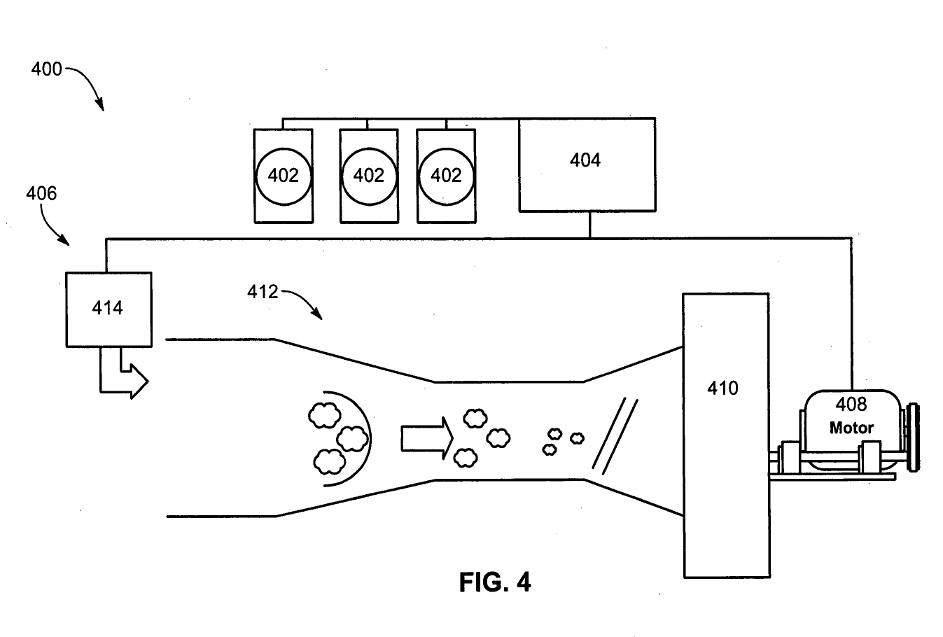

Referring to FIG. 4, a sterilization and monitoring system 400 is shown wherein one or more sensors 402 are located throughout a building structure. The system 400 has particular application with terrorist threats to heavy traffic buildings, such as train stations or airports, and high profile buildings, such as government buildings. Each sensor 402 is in electrical communication with a system computer 404. The sensors 402 detect the presence of biological hazards, such as anthrax or other biological weapons. The system 400 includes an air purification system 406 that may operate continuously at a reduced speed to ensure day-to-day air quality. The air purification system 406 may be accelerated during a hazardous situation to ensure sterilization. Although the system 400 illustrates an air purification system of FIG. 1, one of skill in the art will appreciate that other air purification systems of the present invention may also be used.

Upon detection of a biological hazard, the sensors 402 signal the system computer 404 accordingly. The system computer 404 is in electrical communication with the motor 408 to drive the motor 408 at sufficient speed to ensure sterilization of contaminated air. The motor 408 propels an airflow generator 410 to drive air through a venturi 412. The system computer 404 may further be in electrical communication with a heat source 414 to generate heat and advance sterilization. The system computer 404 further provides audible and visual warning of a biological hazard to signal evacuation. After evacuation, one or more air purification systems 406 continue to operate within the sealed and evacuated building until sterilization is complete. The system computer 404 continues to monitor the air quality to determine if the biological hazard has been eliminated.

Cell structure resiliency varies and air purification systems can increase cell destruction power by increasing the RPMs of the airflow generator. The increased airflow provides for more devastating shock waves, increased friction, and dramatic changes in pressure. A heat source may increase heat input to further accelerate cell destruction. Thus, the air purification systems may vary airflow velocity and heat to destroy resistant cells, such as anthrax, SARS, and other viruses.

The air purification systems require relatively little power to operate and may run continuously. As air purification systems are processing and sterilizing air rather than solids or liquids, they are unlikely to jam. Furthermore, the structure of an air purification system is simple which reduces the likelihood of mechanical failure. There is little wear on an airflow generator, and balancer equipment is unnecessary.

The air purification systems disclosed herein may be employed and disposed within buildings or vehicles as needed. Air purification systems are scalable so that they may be sized based on available space and the volume of air to sterilize. The air purifiers have application in buildings with increased infectious risk, such as hospitals, other medical facilities, child care facilities, immigration facilities, and the like. Air purification systems may be located throughout such a building and operated in conjunction with the HVAC. In one application, a sterilization room may be sealed with air circulating through an air purification system. The sterilization room may be used with travelers during an outbreak of contagious diseases, for surgery, and for clean room laboratories and fabrication.

In passenger vehicles, such as a train or an airplane, one or more air purification systems may run continuously during travel. The spread of disease is greatly reduced as air is sterilized and re-circulated into the vehicle. The number of air purification systems varies depending on air volume to be sterilized and the size of an air purifier. An air purification system may be retrofitted to the existing ventilation systems of airplanes or trains. It is anticipated that one cubic foot of space will accommodate an air purification system of sufficient size to process the air of an average commercial airliner in 2.5 hours. During a flight, all the air may be sterilized and replenished. If needed, the size of an air purification system may be increased to process more air for larger airplanes or for shorter flights.

The embodiments herein disclose a mechanical sterilization apparatus that is reliable and efficient to operate. With speeds approaching supersonic or greater, an air purification system compresses, heats, and applies shockwaves to disrupt cell structure. An air purification system may be used in any number of situations where harmful biological cells must be sterilized.

The above description of illustrated embodiments of the invention is not intended to be exhaustive or to limit the invention to the precise forms disclosed. While specific embodiments of, and examples for, the invention are described herein for illustrative purposes, various equivalent modifications are possible within the scope of the invention, as those skilled in the relevant art will recognize. These and other modifications can be made to the invention in light of the above-detailed description.

1. An air purification system for sterilizing biological cells, comprising:
a venturi, including a converging portion, a throat, and a diverging portion; and
an airflow generator in communication with the venturi to generate an airflow through the venturi and towards the airflow generator, wherein biological cells within the airflow are subjected to compression and shockwaves as the biological cells pass through the venturi.

2. The air purification system of claim 1, further comprising a heat generator in communication with the venturi to provide heat to the airflow.

3. The air purification system of claim 1, further comprising an inlet tube coupled to the converging portion.

4. The air purification system of claim 3, further comprising an inlet device coupled to the inlet tube, the inlet device having a longitudinal axis substantially perpendicular to a longitudinal axis of the inlet tube, the inlet device including opposing first and second apertures.

5. The air purification system of claim 1, wherein the airflow generator is coupled to the diverging portion of the venturi.

6. The air purification system of claim 1, wherein the airflow generator includes,
a housing,
a plurality of radially extending blades, and
an outlet coupled to the housing through which air exits the housing.

7. An air purification system for sterilizing biological cells, comprising:
a venturi, including a converging portion, a throat, and a diverging portion; and
an airflow generator coupled to the converging portion of the venturi and to generate an airflow through the venturi and away from the airflow generator, wherein biological cells within the airflow are subjected to compression and shockwaves as the biological cells pass through the venturi.

8. The air purification system of claim 7, further comprising a heat generator in communication with the venturi to provide heat to the airflow.

9. The air purification system of claim 7, wherein the airflow generator includes,
a housing,
a plurality of radially extending blades, and
an inlet coupled to the housing through which air enters the housing.

10. A method of sterilizing biological in an airflow, comprising:
placing an airflow generator in communication with a venturi;
the airflow generator generating an airflow including biological cells; and
the airflow traversing through the venturi and towards the airflow generator and thereby subjecting the biological cells to compression and shockwaves.

11. The method of claim 10, further comprising coupling an inlet tube to the venturi such that the airflow traverses through the inlet.

12. The method of claim 11, further comprising coupling an inlet device to the inlet tube such that a longitudinal axis of the inlet device is substantially perpendicular to a longitudinal axis of the inlet tube, the inlet device including opposing first and second apertures.

13. The method of claim 10, further comprising placing a heat generator in communication with the venturi to provide heat to the airflow.

14. The method of claim 10, wherein placing an airflow generator in communication with a venturi includes coupling the airflow generator to the diverging portion of the venturi.

15. A method of sterilizing biological cells in an airflow, comprising:
placing an airflow generator in communication with a venturi;
the airflow generator generating an airflow including biological cells; and
the airflow traversing away from the airflow generator and through the venturi and thereby subjecting the biological cells to compression and shockwaves.

16. The method of claim 15, further comprising placing a heat generator in communication with the venturi to provide heat to the airflow.

17. The method of claim 15, wherein placing an airflow generator in communication with a venturi includes coupling the airflow generator to the converging portion of the venturi.

18. A sterilization and monitoring system for detecting and destroying biological cells in air, comprising:
a plurality of sensors, each sensor to detect biological cells and generate a signal indicative of the presence of biological cells;
a computer in electrical communication with the sensors and to receive the signal; and
an air purification system including,
a venturi, including a converging portion, a throat, and a diverging portion,
an airflow generator in communication with the venturi to generate an airflow through the venturi, wherein biological cells within the airflow are subjected to compression and shockwaves as the biological cells pass through the venturi, and
a motor coupled to the airflow generator,
wherein the computer is in electrical communication with the motor to direct operation of the airflow generator.

19. The sterilization and monitoring system of claim 18, further comprising a heat generator in communication with the venturi and wherein the computer is in electrical communication with the heat generator to direct operation of the heat generator.

20. The sterilization and monitoring system of claim 18, wherein the airflow generator directs an airflow towards the airflow generator.

21. The sterilization and monitoring system of claim 18, wherein the airflow generator directs an airflow away from the airflow generator.

22. The sterilization and monitoring system of claim 18, further comprising an inlet tube coupled to the converging portion.

23. The sterilization and monitoring system of claim 22, further comprising an inlet device coupled to the inlet tube, the inlet device having a longitudinal axis substantially perpendicular to a longitudinal axis of the inlet tube, the inlet device including opposing first and second apertures.

24. The sterilization and monitoring system of claim 18, wherein the airflow generator is coupled to the diverging portion of the venturi.
